(19) United States
(12) Patent Application Publication
(10) Pub. No.: US 2023/0021788 A1
(43) Pub. Date: Jan. 26, 2023

(54) MULTI-LAYER DRUG DELIVERY APPARATUS

(71) Applicant: Acclarent, Inc., Irvine, CA (US)

(72) Inventors: Jetmir Palushi, Irvine, CA (US); Fatemeh Akbarian, Rancho Palos Verdes, CA (US); Itzhak Fang, Irvine, CA (US); Athanasios Papadakis, Newport Beach, CA (US); Madison K. Vanosdoll, Cincinnati, OH (US)

(21) Appl. No.: 17/838,571

(22) Filed: Jun. 13, 2022

Related U.S. Application Data
(60) Provisional application No. 63/221,581, filed on Jul. 14, 2021.

(51) Int. Cl. A61M 31/00

(52) U.S. Cl. CPC A61M 31/00; A61M 2210/0662; A61M 2210/0618; A61M 2210/065

(57) ABSTRACT

Some target anatomies within the ear, nose and/or throat of a patient may be difficult to access and treat. To provide a means of treating such target anatomies over time, drug delivery devices that are sized to be positioned within a naturally occurring or man-made anatomical cavity or passageway are preloaded with active agent(s). The drug delivery devices are affixed directly to, or in the vicinity of, a target anatomy. Once affixed, the drug delivery devices are configured to deliver active agent(s) at desired dosage(s) to the target anatomy through controlled elution of the active agent(s) as various structural features of the drug delivery devices are bio-eliminated.

### PRIORITY

This application claims priority to U.S. Provisional Pat. App. No. 63/221,581, entitled “Multi-Layer Drug Delivery Apparatus,” filed Jul. 14, 2021, the disclosure of which is incorporated by reference herein, in its entirety.

### BACKGROUND

Natural openings in the ears, nose and throat may offer easy access to only some internal structures. The labyrinthine anatomy and physiology of the ears, nose and throat may make it difficult, if not impossible, to deliver active agents to targeted treatment sites deep inside of these anatomical structures, without the use of significant medical interventions, including via insertion of catheters and needles through man-made openings in the soft tissue and/or bones of a patient's cranium. Sufficient treatment of ear, nose and throat disorders may warrant multiple applications of one or more active agents to a targeted treatment site over an extended period of time, further increasing complexity of patient care.

There remains a need for a device and method that will allow for prolonged treatment of ear, nose and throat disorders, particularly in areas of these anatomical structures that warrant medical intervention to apply active agents thereto. It may be desirable for such a device and method to be deployable in a paranasal sinus ostium, a eustachian tube, a nasal meatus, another passageway in or adjacent to the nasal cavity, and/or other areas within the ear, nose, and throat. It may be desirable for such a device and method to provide for controlled release of drugs and other therapeutic agents into, or upstream from, targeted treatment sites, giving localized cells sufficient time for absorption of active agents. Furthermore, it may be desirable to separate dosing of active agents by as much as a week or more to ensure that a patient receives the right dosage of one or more active agents at the right times.

While several devices and methods have been made and used to treat ear, nose and throat disorders, it is believed that no one prior to the inventors has made or used the invention described in the appended claims.

### BRIEF DESCRIPTION OF THE DRAWINGS

While the specification concludes with claims that particularly point out and distinctly claim the invention, it is believed the present invention will be better understood from the following description of certain examples taken in conjunction with the accompanying drawings, in which like reference numerals identify the same elements and in which:

FIG. 1 shows a perspective view of an exemplary drug delivery device;

FIG. 2 shows cross-sectional perspective view of the drug delivery device of FIG. 1, taken along line 2-2 of FIG. 1;

FIG. 3 shows a cross-sectional perspective view of an exemplary alternative drug delivery device, formed as a variation of the drug delivery device of FIG. 1;

FIG. 4 shows an enlarged plan view of a portion of an outermost tube of the drug delivery device of FIG. 1;

FIG. 5 shows a perspective cross-sectional view of a layered tube that may form a part of a drug delivery device, the layered tube having a luminal layer and an abluminal layer;

FIG. 6 shows a cross-sectional view of a portion of the layered tube of FIG. 5, taken along line 6-6 of FIG. 5;

FIG. 7 shows a cross-sectional view of a portion of an exemplary variation of the layered tube of FIG. 5, in which the luminal layer comprises a degradable matrix with active particulates embedded in the degradable matrix; and

FIG. 8 shows a cross-sectional view of a portion of another exemplary variation of the layered tube of FIG. 5, in which a luminal layer comprises a degradable matrix with depots embedded in the degradable matrix.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It is to be understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

All percentages, parts and ratios as used herein, are by weight of the total composition, unless otherwise specified. All such weights, as they pertain to listed ingredients, are based on the active level and, therefore, do not include solvents or by-products that may be included in commercially available materials, unless otherwise specified.

All references, including patent applications, patent publications and non-patent literature, that are referred to in the present specification are incorporated by reference herein, unless it is expressly indicated that they are not incorporated by reference herein.

Numerical ranges as used herein are intended to include every number and subset of numbers within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9 and so forth.

All combinations of method or process steps as used herein can be performed in any order, unless otherwise specified or clearly implied to the contrary by the context in which the referenced combination is made. The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

The terms “about” or “approximately” as used herein mean within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “abluminal” as used herein means away from a lumen.

The term “active agent” as used herein means any substance capable of exerting an effect on a patient's tissue and/or bones including, but not limited to, effects selected from therapeutic effects, prophylactic effects and combinations thereof. The term “active agent” as used herein may be broadly construed to include any feasible drugs, prodrugs, proteins, gene therapy preparations, cells, diagnostic agents, contrast or imaging agents, biologicals, etc. Such substances may be in bound or free form, liquid or solid, colloid or other suspension, solution or may be in the form of a gas or other fluid or nan-fluid. Such substance(s) may be in the form of a particulate, or such substances, together with other active agent(s) and/or carriers, may form a particulate (collectively, hereinafter “active particulate” or “active particulates”). For example, in some applications where it is desired to treat or prevent a microbial infection, an active agent to be delivered may comprise a pharmaceutically acceptable salt or dosage form of an antimicrobial agent (e.g., antibiotic, antiviral, antiparacytic, antifungal, etc.), a corticosteroid or other anti-inflammatory (e.g., an NSAID), a decongestant (e.g., vasoconstrictor), a mucous thinning agent (e.g., an expectorant or mucolytic), and/or other compositions. As another merely illustrative example, an active agent may be configured to prevent or modify an allergic response (e.g., an antihistamine, cytokine inhibitor, leucotriene inhibitor, IgE inhibitor, immunomodulatory, etc.). As yet another merely illustrative example, an active agent may include a vasoconstrictor (e.g., for use in patients with chronic rhinitis, etc.).

The term “adluminal” as used herein means towards the lumen.

The term “patient” as used herein means either a human subject or an animal subject.

The term “bio-eliminate” as used herein means to biodegrade, bioabsorb, bioerode and combinations thereof. The term “bio-eliminating” as used herein means biodegrading, bioabsorbing, bioeroding and combinations thereof. The term “bio-eliminated” as used herein means biodegraded, bioabsorbed, bioeroded and combinations thereof. The term “bio-elimination” as used herein means biodegradation, bioabsorption, bioerosion and combinations thereof. The term “bio-eliminable” means capable of being bio-eliminated such as via biodegradation, bioabsorption, bioerosion and combinations thereof. Biodegradation may include chemical cleavage of covalent bonds resulting in decrease in mechanical properties, fragments that may dissolve, metabolize (erosion and degradation are independent although degradation can lead to erosion), etc. Bioabsorption may include polymer or degradation products removed by cellular activity. Bioerosion may include changing from non-water soluble to a water-soluble material (e.g., bulk erosion, in which water influx rate being greater than degradation rate may lead to rapid decrease in mechanical properties and fast breakdown; as compared to surface erosion where polymer hydrolysis is faster than water penetration resulting in gradual decrease in thickness, maintenance of mechanical properties).

The terms “ear, nose and/or throat disorder” and “ENT disorder” are used interchangeably herein to mean any condition that adversely affects the function of the ear, nose and/or throat of a patient.

The term “treatment” as it is used in reference to an ENT disorder, means prevention, reduction, delay, stabilization, elimination and combinations thereof. Some exemplary treatments further include repairing damage caused by an ENT disorder and/or damage caused by mechanical intervention relating to the ear, nose and/or throat.

Internal anatomical structures of the ear, nose and throat may be difficult to access for a physician, and difficult, if not impossible, to access by a patient. Accordingly, it can be difficult to treat target anatomies within the ear, nose and/or throat of a patient, particularly if such treatment includes repeated and/or prolonged delivery of active agent(s) to a target anatomy within the ear, nose or throat of the patient.

Exemplary drug delivery devices in accordance with the present disclosure are sized such that they may be positioned within a naturally occurring or man-made anatomical cavity or passageway such as a nostril, nasal cavity, meatus, ostium, interior of a sinus, etc. Once positioned within the naturally occurring or man-made anatomical cavity or passageway, exemplary drug delivery devices may be affixed directly to, or in the vicinity of, a target anatomy to be treated. Once exemplary drug delivery devices are in place, they may treat anatomy nearby to, and/or anatomy downstream from, a local area of the drug delivery device through controlled elution of one or more active agents from the drug delivery devices.

The duration of treatment of a target anatomy within a patient's ear, nose or throat may depend upon an intended function of the exemplary drug delivery device after it is in place within the patient's body. Thus, the presence of an exemplary drug delivery device within a patient's ear, nose or throat may be necessary for a specified period of time, after which the presence of the exemplary drug delivery device within the patient's body is no longer needed. Since exemplary drug delivery devices may be located in hard to access regions of a patient's ear, nose or throat, exemplary drug delivery devices may be configured for bio-elimination, such that over time they completely disappear from the bodily region into which exemplary drug delivery devices have been deployed. Accordingly, exemplary drug delivery devices may be fabricated from one or more materials that are biodegradable, bioabsorbable, bioerodable and combinations thereof

A protocol for treatment of a target anatomy may require or otherwise warrant application of one or more active agents, at one or more dosages, at different times and/or for different durations of time. Since regions of a patient's ear, nose or throat may be difficult to access, exemplary drug delivery devices may be preloaded with one or more active agents at desired dosage(s). Exemplary drug delivery devices may be configured to release the one or more active agents into a patient's body as various structural features of the drug delivery devices are bio-eliminated. Accordingly, an exemplary drug delivery device may be fabricated from materials that are characterized by different rates of bio-elimination.

In general, an exemplary drug delivery device includes a plurality of tubes coaxially arranged, one inside another. Voids may be formed between an inner surface of a first tube and an outer surface of a second tube located inside the first tube. Spaces between tubes may provide reservoirs into which active agent(s) may be preloaded. In addition, structural elements, such as walls of the plurality of tubes, may comprise components that lend strength thereto, while also providing additional means of preloading exemplary drug delivery devices. These and other aspects of exemplary drug delivery devices are described hereinbelow.

### I. Exemplary Drug Delivery Devices

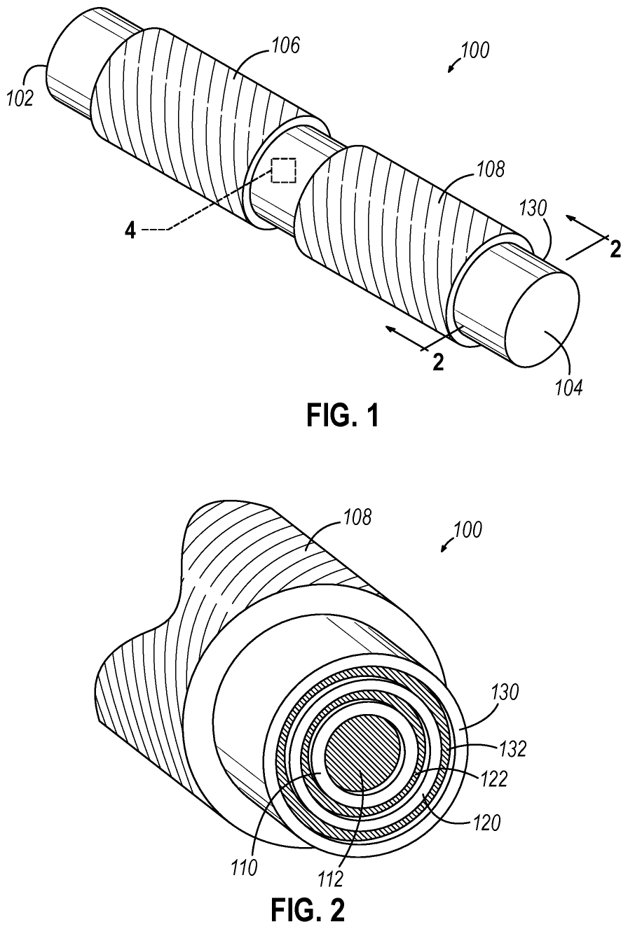

FIGS. 1-2 show an exemplary drug delivery device (100). The exemplary drug delivery device (100) comprises an elongated tubular structure, with each ends (102, 104) of the drug delivery device (100) being sealed. Drug delivery device (100) of the present example comprises three tubes (110, 120, 130) that are arranged coaxially and concentrically with each other, as shown in FIGS. 1-2. Other variations may include more than three tubes or fewer than three tubes. In the present example, each tube (110, 120, 130) is formed of a single layer of material. Some variations may comprise one or more tubes that are each constructed from multiple layers of material. Examples of how multiple layers of material may be used to form tubes (110, 120, 130) will be described in greater detail below.

As shown in FIG. 2, drug delivery device (100) is configured such that tubes (110, 120, 130) are radially spaced apart from each other. Tubes (110, 120, 130) thus cooperate to define lumens (112, 122, 132). Starting from the outside of drug delivery device (100) and moving inwardly, second tube (120) is disposed within a lumen (132) of third tube (130), first tube (110) is disposed within a lumen (122) of second tube (120), and the lumen (112) of first tube (110) forms a core of drug delivery device (100). An outside (or abluminal) wall of third tube (130) forms an outer wall of drug delivery device (200, 300). Some variations of drug delivery device (100) may comprise additional tubes similarly concentrically arranged with respect to each other, such that some variations may have any useful number of layers of tubes defining lumens, such that the drug delivery devices are configured to deliver active agent(s) and are bio-eliminable.

While only the cylindrical sidewalls of tubes (110, 120, 130) are shown, some versions of tubes (110, 120, 130) may have respective closed ends that are longitudinally spaced apart from each other. For instance, ends (102, 104) shown in FIG. 1 may represent integral ends of tube (130); and tube (120) may have corresponding ends (not shown) that are longitudinally spaced from ends (102, 104); and tube (110) may have corresponding ends (not shown) that are longitudinally spaced from the ends of tube (120). Tubes (110, 120, 130) may thus be configured as a set of, close-ended cylinders. Lumen (132) may communicate with a space defined between the outside of the ends of tube (120) and the inside of ends (102, 104) of tube (104). Similarly, lumen (122) may communicate with a space defined between the outside of the ends of tube (110) and the inside of the ends of tube (120). The wall thickness of the ends of tubes (110, 120, 130) may be the same as the wall thickness of the respective cylindrical sidewalls of tubes (110, 120, 130). Alternatively, the wall thickness of the ends of tubes (110, 120, 130) may be greater than or less than the wall thickness of the respective cylindrical sidewalls of tubes (110, 120, 130). As yet another variation, ends (102, 104) may be configured such that ends (102, 104) are not bio-eliminable; and such that ends (102, 104) remain in place while tubes (110, 120, 130) bio-eliminate. Alternatively, ends (102, 104) may be configured such that ends (102, 104) do not bio-eliminate until after tube (110) has bio-eliminated.

In the present example, each lumen (112, 122, 132) contains a respective active agent (as represented in FIG. 2 by the diagonal lines interior to drug delivery device (100)). To provide for controlled release of active agent(s) over time from drug delivery device (100): third (outer-most) tube (130) is made from a material that is configured to be bio-eliminated at a first bio-elimination rate, B₁; second tube (120) is made from a material that is configured to be bio-eliminated at a second bio-elimination rate, B₂; and first tube (110) is made from a material configured to be bio-eliminated at a third bio-elimination rate, B₃. When B₁ is faster than B₂, and B₂ is faster than B₃, drug delivery device (100) successively releases a first active agent from lumen (112), then releases a second active agent from lumen (122) and then releases a third active agent from lumen (132) over a period of time. In some versions, third tube (130) has the fastest bio-elimination rate B₁, such that third tube (130) is the first tube (110, 120, 130) to bio-eliminate. The active agent in lumen (132) may thus be the first active agent to be delivered from drug delivery device (100).

In some such versions (or in other versions), first tube (110) has the slowest bio-elimination rate B₃, such that first tube (110) is the last tube (110, 120, 130) to bio-eliminate. The active agent in lumen (212) may thus be the last active agent to be delivered from drug delivery device (100). Successive release of active agents from drug delivery devices such as drug delivery device (100) may occur over a period of seconds, minutes, hours, days or weeks. By way of example only, delivery of one or more active agents to the target anatomical structure from lumens (112, 122, 132) may occur in succession. By way of example only, delivery of active agents to the target anatomical structure may comprise successive delivery of active agent to the target anatomical structure from the outermost lumen (132) during a first period of time, delivery of active agent to the target anatomical structure from the intermediate lumen (122) during a second period of time and delivery of active agent to the target anatomical structure from the innermost lumen (112) during a third period of time. In some versions, the active agent from lumen (132) may be delivered during a first week; the active agent from lumen (122) may be delivered during a second week following the first week; and the active agent from lumen (132) may be delivered during a third week following the first and second weeks.

Drug delivery device (100) of the present example further includes a pair of integral stents (106, 108). Stents (106, 108) are configured to serve as attachment features that may be used to secure drug delivery device (100) in place within a patient's ear, nose or throat. Stents (106, 108) are located around a periphery of outside (or abluminal) wall of third tube (130) and are longitudinally spaced apart from each other. As shown in FIG. 1, stents (106,108) are initially in a collapsed configuration. After drug delivery device (100) is located in a desired position within a patient's ear, nose or throat, stents (106, 108) are deployed such that they are in an expanded configuration that contacts a patient's tissue and/or bone and holds drug delivery device (100) in place. In some versions, stents (106, 108) are self-expanding. For instance, stents (106, 108) may be resiliently biased to expand; and stents (106, 108) may be maintained in a compressed configuration by a deployment sheath (not shown) until drug delivery device (100) is deployed at the target site. Once drug delivery device (100) is located at the target site, the sheath may be removed to allow stents (106, 108) to expand. In some other variations, the expansion of stents (106, 108) is thermally activated (e.g., by the body temperature of the patient at the target site, etc.). Alternatively, stents (106, 108) may transition from a non-expanded state (pre-deployment) to an expanded state (post-deployment) in any other suitable fashion as will be apparent to those skilled in the art in view of the teachings herein.

While stents (106, 108) are used in the present example, any other suitable kind(s) of attachment features may be used, including but not limited to stents, sutures, adhesives, self-expanding portions that are configured to grip bone and/or tissue, barbs, clips, screws, a suction apparatus, and combinations thereof. In some variations, more than one type of attachment portion or a combination of two or more attachment portions may be utilized. In versions and variations of drug delivery device (100) that include an attachment feature such as stents (106, 108), etc., the attachment feature may comprise material that is configured to be bio-eliminated, so that it completely disappears from the bodily region into which the drug delivery device has been deployed.

In some versions, stents (106, 108) are secured to tube (110) such that stents (106, 108) continue to secure drug delivery device (100) in the targeted cavity or anatomical passageway even after tubes (120, 130) have bio-eliminated. Other suitable ways in which stents (106, 108) and/or other attachment features may maintain drug delivery device (100) in the targeted cavity or anatomical passageway throughout bio-elimination of tubes (110, 120, 130) will be apparent to those skilled in the art in view of the teachings herein.

Active agent(s) may be preloaded in lumens (112, 122, 132) drug delivery device (100) in any suitable fashion and combination. For example, in some versions, each lumen (112, 122, 132) may be preloaded such that each lumen (112, 122, 132) contains one or more active agents, which may be the same or different from each other, and/or may be present in each of the lumens (112, 122, 132) in the same or in different concentrations. In the example of drug delivery device (100) shown in FIGS. 1-2, lumens (112, 122, 132) each contain the same drug in the same dosage (as represented in FIG. 2 by the diagonal lines interior to drug delivery device (100)).

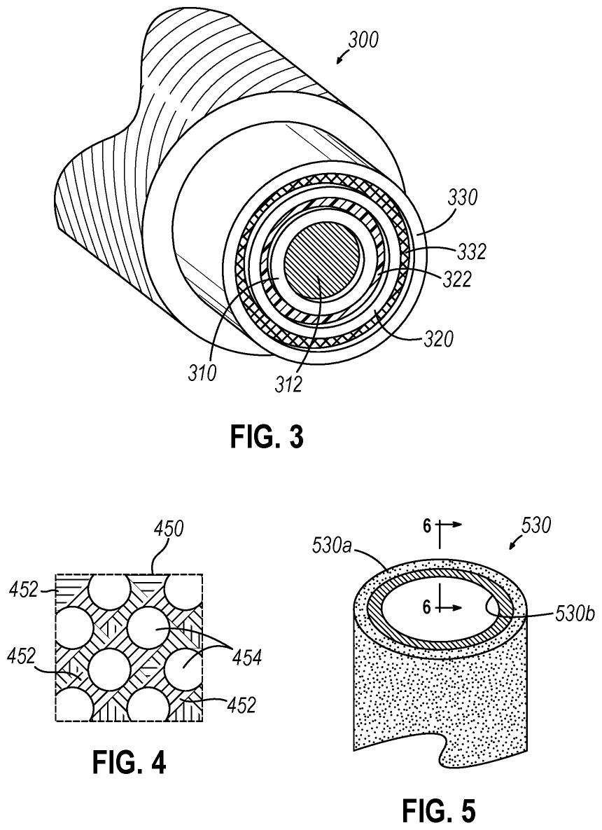

FIG. 3 shows an exemplary alternative drug delivery device (300) that is substantially similar to drug delivery device (100). In particular, drug delivery device (300) of this example has structural features (310, 312, 320, 322, 330, 332) that are identical to the structural features (110, 112, 120, 122, 130, 132) of drug delivery device (100). However, unlike the lumens (112, 122, 132) of drug delivery device (100), the lumens (312, 322, 332) of drug delivery device (300) each contain a different active agent or the same active agent in different concentrations (as represented in FIG. 3 by the diagonal lines and cross-hatchings interior to drug delivery device (300)). Any useful combination of one or more active agents in one or more dosages may be preloaded into lumens (112, 122, 132, 312, 322, 332) of drug delivery devices (100, 300). In some variations, each lumen (112, 122, 132, 312, 322, 332) is loaded with a polymeric carrier that includes one or more active agent(s).

In addition to lumens of drug delivery devices (100, 300) being preloaded with active agent(s), some variations of drug delivery devices (100, 300) may include comprise variations of tubes (110, 120, 130, 310, 320, 330) and/or other structural components (e.g., stents 106, 108) or other attachment features) that are made from material that is coated with active agent(s) and/or material that has one or more active agents embedded therein. For example, FIG. 4 shows an enlarged view of a portion of material (450) that may be used for form one or more of tubes (110, 120, 130, 310, 320, 330). As can be seen in FIG. 4, material (450) comprises a bio-eliminable matrix (452), with a plurality of active particulates (454) embedded therein.

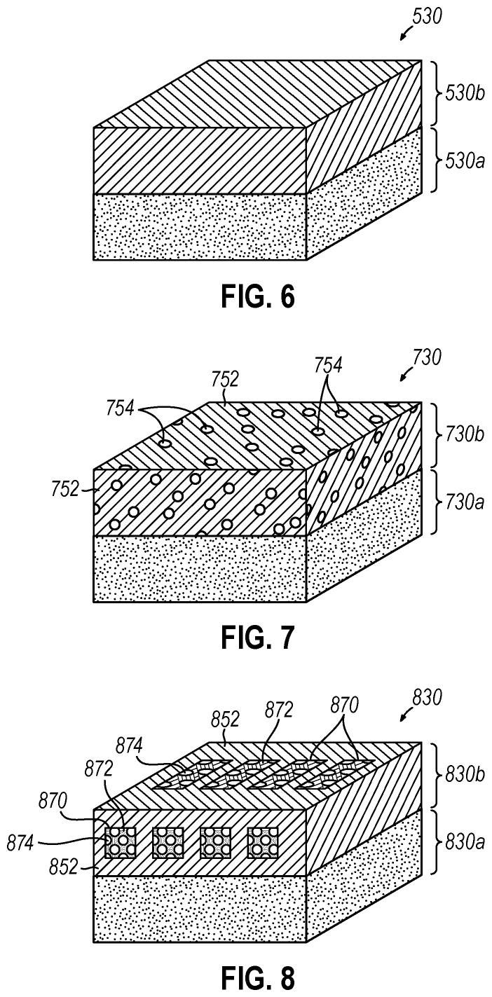

FIG. 5, shows an exemplary alternative tube (530) that may be used in place of any one or more of (110, 120, 130, 310, 320, 330). Tube (530) of this example has two layers made of different materials, including an abluminal layer (530a) and an adluminal layer (530b). FIG. 6 shows an enlarged cross-sectional view of layers (630a, 630b) in direct apposition with each other. Other configurations of multilayered tubes may be of use, including those that further comprise one or more middle layers between luminal and abluminal layers.

FIG. 7 shows an enlarged cross-sectional view of layer configuration for an exemplary alternative tube (730) that may be used in place of any one or more of tubes (110, 120, 130, 310, 320, 330). In tube (730) of this example, adluminal layer (730b) comprises a bio-eliminable matrix (752) with plurality of active particulates (754) embedded in bio-eliminable matrix. Once a version of drug delivery device (100) having a bio-eliminable matrix (452, 752) is implanted into a patient's ear, nose or throat, bio-eliminable matrix (452, 752) may degrade, dissolve and/or erode, allowing particulate active agents (454, 754) to be released from drug delivery device (100) into the patient's body.

To fabricate a coating comprising active agent(s), a polymer or a blend of polymers may be applied to a material either before or after a tube (110, 120, 130, 310, 320, 330, 530, 730, 830) is made, using techniques known to those skilled in the art. A coating comprising active agent(s) may be applied to the material or to a finished tube (110, 120, 130, 310, 320, 330, 530, 730, 830), for example, by immersing the material or finished tube (110, 120, 130, 310, 320, 330, 530, 730, 830) in a coating material including a polymer, solvent and active agent(s); or by spraying the coating material including active agent(s) onto the material or tube (110, 120, 130, 310, 320, 330, 530, 730, 830). Some variations of tubes (110, 120, 130, 310, 320, 330, 530, 730, 830) may comprise active agent(s) that are dispersed within a polymer during extrusion of the tubes (110, 120, 130, 310, 320, 330, 530, 730, 830). Additionally, or alternatively, a coating comprising active agent(s) may be applied to and/or incorporated into a drug delivery device (100, 300) after the drug delivery device (100, 300) is assembled.

By using releasably embedded particles in drug delivery devices (100, 300), treatment of

ENT disorders may occur downstream from an anatomical region where drug delivery device (100, 300) is deployed. Some variations of drug delivery devices (100, 300) comprise one or more tubes having depots embedded therein. Depots may be configured to have a slower or faster rate of bio-elimination than material surrounding them. FIG. 8 shows an enlarged cross-sectional view of layer configuration for an exemplary alternative tube (830) that may be used in place of any one or more of tubes (110, 120, 130, 310, 320, 330). In tube (830) of this example, adluminal layer (830b) comprises bio-eliminable matrix (852) with a plurality of depots (870) embedded therein. Depots (870) may be at least partially filled by a bio-eliminable material (872) with a plurality of active particulates (874) embedded therein. Depots (870) may provide another means of controlling timing of release of active agents from drug delivery devices that integrate one or more of tubes (830) in place of one or more of tubes (110, 120, 130, 310, 320, 330).

To control release of active agent(s) over time, the various features of drug delivery devices (100, 300) as described herein may be configured to have different bio-elimination rates. For example, each tube (110, 120, 130, 310, 320, 330, 530, 730, 830) of a drug delivery device (100, 300), each bio-eliminable matrix (452, 752, 852), each depot (870), and/or each attachment feature (e.g., stents (106, 108), etc.) may comprise components that provide the various features of drug delivery devices (100, 300) with discrete, staged bio-elimination rates.

In the present example, drug delivery device (100, 300) is configured as a solid structure with a solid core and closed ends (102, 104). Thus, in the present example, air or other fluids do not pass through the anatomical passageway in which drug delivery device (100, 300) is disposed. Even as drug delivery device (100, 300) degrades or otherwise bio-eliminates while drug delivery device (100, 300) is disposed in the patient, the solid configuration of drug delivery device (100, 300) may continue to prevent air or other fluids from passing through the inner core region (e.g., the region occupied by tube (110, 310) and the active agent within lumen (112, 312)). In some variations, drug delivery device (100, 300) may be configured to allow air or other fluids to pass around the exterior of the deployed drug delivery device (100, 300) (e.g., while still not allowing air or other fluids from passing through the inner core region).

### II. Method of Use

Treatment of an ENT disorder with drug delivery devices (100, 300) may involve delivery and attachment of the drug delivery devices (100, 300). Some exemplary means of delivery and deployment of drug delivery devices (100, 300) may involve positioning an drug delivery device (100, 300) about one end of a catheter, inserting that end of the catheter through a naturally occurring or man-made anatomical cavity or passageway, advancing the catheter in the anatomical cavity or passageway to a desired treatment location, attaching the drug delivery device (100, 300) to tissue and/or bone located at a treatment location, and removing the catheter from the anatomical cavity or passageway. The drug delivery device (100, 300) is left inside the anatomical cavity or passageway to release one or more active agent(s) in a controlled fashion over time to target anatomy at and/or downstream of the treatment location. Such delivery may include staged delivery of different active agents in a particular sequence, with each different active agents being delivered at a different respective time and/or for a different respective duration. The drug delivery device (100, 300) disappears over time through bio-elimination, thus obviating the need to remove the drug delivery device (100, 300) through a further medical procedure.

### III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A drug delivery device configured for controlled delivery of one or more active agents to a targeted anatomical structure inside a patient's ear, nose or throat, the drug delivery device being configured to be bio-eliminated from the patient's body, wherein the drug delivery device has an elongated tubular structure being sealed closed at each of two terminuses of the tubular structure, the drug delivery device further comprising: (a) a first tube defining a first lumen, wherein the first tube is characterized by a first bio-elimination rate, B₁; (b) a second tube defining a second lumen, wherein the second tube is characterized by a second bio-elimination rate, B₂; and (c) a third tube defining a third lumen, wherein the third tube is characterized by a third bio-elimination rate, B₃; wherein: (i) the first, second and third tubes are concentrically arranged such that: (A) the first tube is disposed within the second lumen of the second tube, and (B) the second tube is disposed within the third lumen of the third tube, (ii) the first, second and third lumens each contain an active agent, and (III)the first, second and third bio-elimination rates (B₁, B₂, B₃) are different.

### EXAMPLE 2

The drug delivery device of example 1, wherein each of the first, second and third tubes are configured as a set of nested, close-ended cylinders.

### EXAMPLE 3

The drug delivery device of any of examples 1 through 2, wherein: (a) the drug delivery device is configured as a solid structure, and (b) at least one of the first, second or third lumens comprises a solid core.

### EXAMPLE 4

The drug delivery device of any of examples 1 through 3, further comprising an attachment device affixed to an exterior of the third tube, wherein the attachment portion is configured to releasably attach the drug delivery device to bone and/or tissue inside the patient's ear, nose or throat.

### EXAMPLE 5

The drug delivery device of example 4, wherein the attachment portion is selected from: a stent, a suture, an adhesive, a self-expanding portion that is configured to grip bone and/or tissue, barb(s) and combinations thereof.

### EXAMPLE 6

The drug delivery device of any of examples 1 through 5, wherein the first, second and third lumens contain the same active agent.

### EXAMPLE 7

The drug delivery device of any of examples 1 through 6, wherein the first, second and third lumens each contains different active agents.

### EXAMPLE 8

The drug delivery device of any of examples 1 through 7, wherein: (a) the third bio-elimination rate, B₃, is faster than the second bio-elimination rate, B₂; and (b) the second bio-elimination rate, B₂, is faster than the first bio-elimination rate, B₃.

### EXAMPLE 9

The drug delivery of any of examples 1 through 8, wherein at least one of the first, second or third tubes is a layered tube comprising: (a) an adluminal layer, and (b) an abluminal layer; wherein the adluminal layer and the abluminal layer each have a different bio-elimination rate.

### EXAMPLE 10

The drug delivery device of example 9, wherein at least one of the adluminal layer or the abluminal layer comprises: (a) a bio-eliminable matrix; and (b) a plurality of particles, wherein: (i) the particles are embedded in the bio-eliminable matrix, and (ii) the particles are configured to be released from the bio-eliminable matrix as the bio-eliminable matrix degrades, is absorbed by the patient's body and/or bioerodes.

### EXAMPLE 11

The drug delivery device of example 9, wherein at least one of the adluminal layer or the abluminal layer comprises: (a) a bio-eliminable matrix; and (b) a plurality of depots having a degradable material at least partially filling each of the plurality of depots; wherein: (i) each of the plurality of depots are embedded in the bio-eliminable matrix, and (ii) each of the plurality of the depots have a faster bio-elimination rate than the bio-elimination rate of the bio-eliminable matrix.

### EXAMPLE 12

The drug delivery device of example 11, wherein one or more of the plurality of depots contain particles that are configured to be released when the one or more depots are bio-eliminated.

### EXAMPLE 13

The drug delivery device of example 12, wherein the one or more of the plurality of particles contain an active agent.

### EXAMPLE 14

The drug delivery device of any of examples 1 through 13, wherein at least one of the first, second or third tubes comprises: (a) a matrix that is biodegradable, bioerodable, bioabsorbable and combinations thereof; and (b) a plurality of particles loaded with an active agent, wherein: (i) the particles are embedded in the matrix, and (ii) the particles are configured to be released from the matrix when the matrix biodegrades, bioerodes, is bioabsorbed and combinations thereof.

### EXAMPLE 15

A method for controlled delivery of one or more active agents to a target anatomical structure inside a patient's ear, nose or throat, the method comprising inserting a drug delivery device into an ostium and/or a cavity inside the patent's ear, nose or throat, wherein the drug delivery device has an elongated tubular structure being sealed closed at each of two terminuses of the tubular structure, the drug delivery device further comprising: (a) a first tube defining a first lumen, wherein the first tube is characterized by a first bio-elimination rate, B1; (b) a second tube defining a second lumen, wherein the second tube is characterized by a second bio-elimination rate, B2; and (c)a third tube defining a third lumen, wherein the third tube is characterized by a third bio-elimination rate, B3; wherein: (i) the first, second and third tubes are concentrically arranged such that: (A) the first tube is disposed within the second lumen of the second tube, and (B) the second tube is disposed within the third lumen of the third tube, (ii) the first, second and third lumens each contain an active agent, and (iii) the first, second and third bio-elimination rates (B1, B2, B3) are different.

### EXAMPLE 16

The method of example 15, further comprising bioelimination of the third tube prior bioelimination of first tube and the second tube.

### EXAMPLE 17

The method of example 15, further comprising in the following order: (a) bio-elimination of the third tube; (b) bio-elimination of the second tube; and (c) bio-elimination of the first tube.

### EXAMPLE 18

The method of any of examples 15 through 17, wherein: (a) the drug delivery device is configured as a solid structure, and (b) at least the first lumen comprises a solid core containing one or more active agents; the method further comprising releasing the one or more active agents from the solid core.

### EXAMPLE 19

The method of any of examples 15 through 18, further comprising successive: (a) delivery of the active agent to the target anatomical structure from the third lumen during a first period of time; (b) delivery of the agent to the target anatomical structure from the second lumen during a second period of time; and (c) delivery of the agent to the target anatomical structure from the first lumen during a third period of time.

### EXAMPLE 20

A drug delivery device configured for controlled delivery of one or more active agents to a targeted anatomical structure inside a patient's ear, nose or throat, the drug delivery device being configured to be bio-eliminated in whole or in part from the patient's body, wherein the drug delivery device is configured as a solid elongated tubular structure comprising: (a) closed ends at each of two terminuses of the solid elongated tubular structure; (b) a plurality of tubes concentrically arranged as a set of nested, close-ended cylinders, wherein: (i) each of the plurality of tubes: (A) defines a lumen containing an active agent, and (B) is characterized by a bio-elimination rate; wherein the bio-elimination rate of each of the plurality of tubes is either the same or different; and (ii) at least one of the plurality of tubes contains a solid core.

### EXAMPLE 21

A method for controlled delivery of one or more active agents to a target anatomical structure inside a patient's ear, nose or throat, the method comprising inserting a drug delivery device according to any of examples 1 through 14 and 20, into an ostium and/or a cavity inside the patent's ear, nose or throat.

### IV. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

We claim:

1. A drug delivery device configured for controlled delivery of one or more active agents to a targeted anatomical structure inside a patient's ear, nose or throat, the drug delivery device being configured to be bio-eliminated from the patient's body, wherein the drug delivery device has an elongated tubular structure being sealed closed at each of two terminuses of the tubular structure, the drug delivery device further comprising:
(a) a first tube defining a first lumen, wherein the first tube is characterized by a first bio-elimination rate, B₁;
(b) a second tube defining a second lumen, wherein the second tube is characterized by a second bio-elimination rate, B₂; and
(c) a third tube defining a third lumen, wherein the third tube is characterized by a third bio-elimination rate, B₃;
wherein:
(i) the first, second and third tubes are concentrically arranged such that:
(A) the first tube is disposed within the second lumen of the second tube, and
(B) the second tube is disposed within the third lumen of the third tube,
(ii) the first, second and third lumens each contain an active agent, and
(iii) the first, second and third bio-elimination rates (B₁, B₂, B₃) are different.

2. The drug delivery device of claim 1, wherein each of the first, second and third tubes are configured as a set of nested, close-ended cylinders.

3. The drug delivery device of claim 1, wherein:
(a) the drug delivery device is configured as a solid structure, and
(b) at least one of the first, second or third lumens comprises a solid core.

4. The drug delivery device of claim 1, further comprising an attachment device affixed to an exterior of the third tube, wherein the attachment portion is configured to releasably attach the drug delivery device to bone and/or tissue inside the patient's ear, nose or throat.

5. The drug delivery device of claim 4, wherein the attachment portion is selected from: a stent, a suture, an adhesive, a self-expanding portion that is configured to grip bone and/or tissue, barb(s) and combinations thereof.

6. The drug delivery device of claim 1, wherein the first, second and third lumens contain the same active agent.

7. The drug delivery device of claim 1, wherein the first, second and third lumens each contains different active agents.

8. The drug delivery device of claim 1, wherein:
(a) the third bio-elimination rate, B₃, is faster than the second bio-elimination rate, B₂; and
(b) the second bio-elimination rate, B₂, is faster than the first bio-elimination rate, B₃.

9. The drug delivery device of claim 1, wherein at least one of the first, second or third tubes is a layered tube comprising:
(a) an adluminal layer, and
(b) an abluminal layer;
wherein the adluminal layer and the abluminal layer each have a different bio-elimination rate.

10. The drug delivery device of claim 9, wherein at least one of the adluminal layer or the abluminal layer comprises:
(a) a bio-eliminable matrix; and
(b) a plurality of particles,
wherein:
(i) the particles are embedded in the bio-eliminable matrix, and
(ii) the particles are configured to be released from the bio-eliminable matrix as the bio-eliminable matrix degrades, is absorbed by the patient's body and/or bioerodes.

11. The drug delivery device of claim 9, wherein at least one of the adluminal layer or the abluminal layer comprises:
(a) a bio-eliminable matrix; and
(b) a plurality of depots having a degradable material at least partially filling each of the plurality of depots;
wherein:
(i) each of the plurality of depots are embedded in the bio-eliminable matrix, and
(ii) each of the plurality of the depots have a faster bio-elimination rate than the bio-elimination rate of the bio-eliminable matrix.

12. The drug delivery device of claim 11, wherein one or more of the plurality of depots contain particles that are configured to be released when the one or more depots are bio-eliminated.

13. The drug delivery device of claim 12, wherein the one or more of the plurality of particles contain an active agent.

14. The drug delivery device of claim 1, wherein at least one of the first, second or third tubes comprises:
(a) a matrix that is biodegradable, bioerodable, bioabsorbable and combinations thereof; and
(b) a plurality of particles loaded with an active agent,
wherein:
(i) the particles are embedded in the matrix, and
(ii) the particles are configured to be released from the matrix when the matrix biodegrades, bioerodes, is bioabsorbed and combinations thereof.

15. A method for controlled delivery of one or more active agents to a target anatomical structure inside a patient's ear, nose or throat, the method comprising inserting a drug delivery device into an ostium and/or a cavity inside the patent's ear, nose or throat, wherein the drug delivery device has an elongated tubular structure being sealed closed at each of two terminuses of the tubular structure, the drug delivery device further comprising:
(a) a first tube defining a first lumen, wherein the first tube is characterized by a first bio-elimination rate, Bi;
(b) a second tube defining a second lumen, wherein the second tube is characterized by a second bio-elimination rate, B₂; and
(c) a third tube defining a third lumen, wherein the third tube is characterized by a third bio-elimination rate, B₃;
wherein:
(i) the first, second and third tubes are concentrically arranged such that:
(A) the first tube is disposed within the second lumen of the second tube, and
(B) the second tube is disposed within the third lumen of the third tube,
(ii) the first, second and third lumens each contain an active agent, and
(iii) the first, second and third bio-elimination rates (B₁, B₂, B₃) are different.

16. The method of claim 15, further comprising bioelimination of the third tube prior bioelimination of first tube and the second tube.

17. The method of claim 15, further comprising in the following order:
(a) bio-elimination of the third tube;
(b) bio-elimination of the second tube; and
(c) bio-elimination of the first tube.

18. The method of claim 17, wherein:
(a) the drug delivery device is configured as a solid structure, and
(b) at least the first lumen comprises a solid core containing one or more active agents;
the method further comprising releasing the one or more active agents from the solid core.

19. The method of claim 17, further comprising successive:
(a) delivery of the active agent to the target anatomical structure from the third lumen during a first period of time;
(b) delivery of the agent to the target anatomical structure from the second lumen during a second period of time; and
(c) delivery of the agent to the target anatomical structure from the first lumen during a third period of time.

20. A drug delivery device configured for controlled delivery of one or more active agents to a targeted anatomical structure inside a patient's ear, nose or throat, the drug delivery device being configured to be bio-eliminated in whole or in part from the patient's body, wherein the drug delivery device is configured as a solid elongated tubular structure comprising:
(a) closed ends at each of two terminuses of the solid elongated tubular structure; and
(b) a plurality of tubes concentrically arranged as a set of nested, close-ended cylinders, wherein:
(i) each of the plurality of tubes:
(A) defines a lumen containing an active agent, and
(B) is characterized by a bio-elimination rate,
wherein the bio-elimination rate of each of the plurality of tubes is either the same or different, and
(ii) the plurality of tubes contains a solid core.
